(19) United States
(12) Patent Application Publication
(10) Pub. No.: US 2007/0282246 A1
(43) Pub. Date: Dec. 6, 2007

(54) Iontosonic-microneedle biosensor apparatus and methods

(75) Inventor: Julian L. Henley, New Haven, CT (US)

Correspondence Address: STERNE, KESSLER, GOLDSTEIN & FOX P.L.L.C., 1100 NEW YORK AVENUE, N.W., WASHINGTON, DC, 20005, US

(73) Assignee: MIT, LLP, Miami, FL (US)

(21) Appl. No.: 11/599,329

(22) Filed: Nov. 15, 2006

Related U.S. Application Data
(60) Provisional application No. 60/810,657, filed on Jun. 5, 2006.

(51) Int. Cl. A61N 1/30

(52) U.S. Cl. 604/19

(57) ABSTRACT

A screening device that screens for the presence or absence of one or more substances in a mammalian body is disclosed. The device includes a plurality of electrodes, and a biosensor assembly having a reference region and at least one sensor region. The reference region includes at least one microneedle and microchannel, and each sensor region includes at least one microneedle and microchannel, and is sensitive to a specific substance. Each sensor microchannel in a respective sensor region includes a specific assay ligand that may bond with the particular substance to be sensed. Also disclosed is a method for the screening for the presence or absence of one or more substances in the mammalian body.

### BACKGROUND OF THE INVENTION

1. Field of the Invention

The present invention relates to biosensor devices and methods.

2. Background Art

The ability to detect substances present in a human body has many important applications. Screening subjects for substance abuse is necessary when trying to ensure a safe environment. Laboratory workers who deal with hazardous materials or airline pilots who fly passenger planes are required to undergo routine drug screening to confirm they are not abusing illicit substances that may impair other's safety. Drug screening has also become necessary in competitive athletics in the hope of maintaining a fair and level competitive playing field. Screening athletes for use of anabolic steroids, growth hormones and other performance enhancing agents has become routine in Olympic, collegiate and professional sports.

Many methods exist for screening subjects for substance use and abuse. Urine analysis and blood analysis are widely-used methods, but require strict oversight, can be uncomfortable for the patient, and require a period of days or weeks before results are available. Urine analysis, for example, requires oversight to confirm that the urine being analyzed is from the subject and not another person, which usually requires temperature measurements. In addition, the density of the urine must be measured to confirm that the urine has not been diluted with water or other fluids before submission. These necessary compliance issues make urine analysis burdensome and can often delay test results.

Microneedle arrays are known in the art for drug delivery, and employ microneedles with lengths of about 10-350 μm in order to enhance permeability of the drug without causing pain to the subject. Also, microneedles have been used in combination with ionophoresis to enhance skin penetration and the sampling of serum transdermally. Retrograde iontophoretic sampling of transdermal serum and or transudes has been used in measurement of glucose and originally used in diagnostic work up of cystic fibrosis.

Reverse ionophoresis has been used to create serum transudation and allow for repeated painless measurement of glucose with a specific electrode mounted outside of the skin.

What is needed is a rapid and painless method of detecting substances in a subject that does not require great oversight, is not burdened by delays of test results, and can function as an early warning screening test.

### BRIEF SUMMARY OF THE INVENTION

An embodiment of the present invention relates to a screening device that screens for the presence or absence of one or more substances in a mammalian body. The device includes a plurality of electrodes, and a biosensor assembly having a reference region and at least one sensor region. The reference region includes at least one microneedle and a microchannel, and each sensor region includes at least one microneedle and microchannel, and is sensitive to a respective substance. Each sensor microchannel in a respective sensor region includes a specific assay ligand that may bond with the particular substance to be sensed.

In another embodiment, the present invention relates to a method of screening a mammal for the presence or absence of a substance, comprising: contacting a biosensor assembly with a portion of the skin of a mammal; extracting a transudate from the skin iontophoretically; contacting the biosensor assembly with the transudate; and determining the presence or absence of the substance in the transudate. The biosensor assembly comprises a reference region and at least one sensor region. The reference region includes at least one microneedle and a microchannel, and each sensor region includes at least one microneedle and microchannel, and is sensitive to the substance. Each sensor microchannel in a respective sensor region includes a specific assay ligand that may bond with the substance to be sensed.

Further embodiments, features, and advantages of the invention, as well as the structure and operation of the various embodiments of the invention are described in detail below with reference to accompanying drawings.

### BRIEF DESCRIPTION OF THE DRAWINGS/FIGURES

The accompanying drawings, which are included to illustrate exemplary embodiments of the invention and are incorporated in and constitute a part of this specification, illustrate embodiments of the invention and together with the description serve to explain the principles of the invention. In the drawings:

FIG. 1 shows a side view of a biosensor array device including microneedles for the screening of a substance in a mammalian body.

FIG. 2 shows a side view of a biosensor array device including microneedles and a differential measurement controller for determining the presence of a substance in the microchannels.

FIG. 3 shows a flowchart for various steps in a method for screening for a substance in a mammalian body.

The present invention will be described with reference to the accompanying drawings. The drawing in which an element first appears is typically indicated by the leftmost digit(s) in the corresponding reference number.

### DETAILED DESCRIPTION OF THE INVENTION

Reference will now be made in detail to the embodiments of the present invention, examples of which are illustrated in the accompanying drawings and examples.

It should be appreciated that the particular implementations shown and described herein are examples of the invention and are not intended to otherwise limit the scope of the present invention in any way. Indeed, for the sake of brevity, conventional electronics, manufacturing, semiconductor devices, and nanotube technologies and other functional aspects of the systems (and components of the individual operating components of the systems) may not be described in detail herein.

Iontophoresis refers to the movement of molecules through a membrane by an electrical field. Iontophoretic drug delivery refers to the delivery of drugs through a subject's skin by the action of an electric field. If the polarity of the electric field is reversed, however, it is possible to extract transudate from the subject's skin. This method is often referred to as “reverse iontophoresis.”

Many critical jobs and endeavors (airline pilots, air traffic control, machinery operators etc.) need to be free of illicit drugs that may affect performance. Current control systems involve random spot drug testing that involves urine samples and/or blood samples. The labeling, transport, packaging and identification of such specimens is subject to further abuses, errors, implementation costs and delays. With the technology described herein a biosensor array is integrated with an electrode to create an apparatus that gives an immediate screen for the presence or absence of a variety of illicit compounds that are subject to recreational abuse at the expense of safety and performance of critical jobs that may affect not only the worker but innocent bystanders.

By introducing surface mounted specific ligands within the walls of the pores of a plurality of microchannels connected to iontophoretic electrodes, immediate reading from each electrode is possible as to the presence or absence of substances that may bind the specific ligands.

In one embodiment, the dispersion electrode has a dual purpose. During the retrograde serum acquisition phase it functions as an iontophoretic driver to sequester the transdermal transudate within the lumen of each pore that is adjacent to the base of microneedle and the electronics behind each electrode will vary the voltage to overcome resistive obstacles yet maintain a steady predetermined current. During the measurement phase, the electrode is removed from the skin and the ligands within such micro lumens have attached to the molecule in question. Such micro lumens now will offer a potentiometric and capacitive dielectric load to the same electrode in contact with such lumens.

In one embodiment of the present invention, during clinical applications such dispersive electrode assembly will develop different voltage potentials at each channel in order to maintain the same current flow through different segments of the skin that exhibited a varying resistive impediment to such flow. Such multichannel configuration can overcome limitations of wide field iontophoretic dispersion. For example, U.S. Pat. No. 5,160,316, issued to the instant inventor and incorporated in its entirety herein by reference, describes the use of a multichannel dispersive electrode. U.S. Pat. No. 5,658,247, issued to the instant inventor, and incorporated herein by reference in its entirety, describes a multichannel ionophoretic driver mounted on same application electrode with ultrasonic elements for enhanced intradermal delivery of therapeutic agents.

Prior single channel electrodes or plurality of electrodes driven by one circuit were plagued by tunneling effects where the preferred current flux occurred through the path of least resistance often leading to blister formation and localized skin breakdown.

In an embodiment of the present invention, when the electrode is removed from the skin with the acquired sample, and placed in contact with a uniform neutral electrode, the activation of the same embedded multichannel iontophoretic electrodes will create a measurable voltage drop between the driving electrodes. The sensor electrodes and/or microchannels encounter a resistive load when they overlie the micro channels that are filled with ligand-antigen complex as compared to reference (calibration) electrodes and/or micro channels that do not contain such obstructing complexes. The same driving circuitry imbedded in the electrode therefore efficiently serves to both acquire the sample and than measure it by virtue of the potential differences between the channels.

The same electrode will now yield measurable data correlating with ligand molecule complexes within such proximal micro channels. To further enhance the accuracy of such a device, the plurality of non-ligand micro lumens is used for self-calibration of such multielectrode readout. It is the unique design of this multichannel ionosonic-microneedle electrode that lends itself to this dual step design of acquisition and immediate readout of integrated biosensor.

Such transdermal device that yields immediate screening information for presence of illicit substances to be followed by blood and urine testing if quantitative data is important. Such device will have significant social value in our complex society where critical jobs need to be monitored for the greater safety of all. A similar device with a biosensor array with ligands/receptors for anabolic steroids may be equally useful for an immediate screen before a competitive athletic event rather than forcing athletes to give up medals or team score points ex post facto when the urine/blood analysis results are completed. Such hand held transdermal device based on multichannel iontophoresis, ultrasound, micro needles and biosensors integrated into a portable/transportable unit is described herein.

Microneedles for use in the present invention can be any type of microneedle including but not limited to a hollow and/or solid microneedles made of plastic, diamond, crystal, silicone, glass or other material. The preferred material will be an inert non conductor or semiconductor of sufficient hardness. In one example, the microneedles are made of silicon. In another example, the microneedles are made of silicon dioxide. In yet another example, the microneedles are made of glass. Microneedles 172 can be any desired length and can have uniform or varying lengths depending upon a particular application. In one example, microneedles have lengths in the range of about 10-350 μm. These example materials and dimensions are illustrative and not intended to limit the present invention. A cross section of bundled fiber optic cables exhibits such an example of micro needles interspersed with micro channels. A bundled cross section of microtubules from heat drawn glass tubing is another example. Porous fiberglass with protruding fibers is yet another manufacturing example of the embodiment described herein.

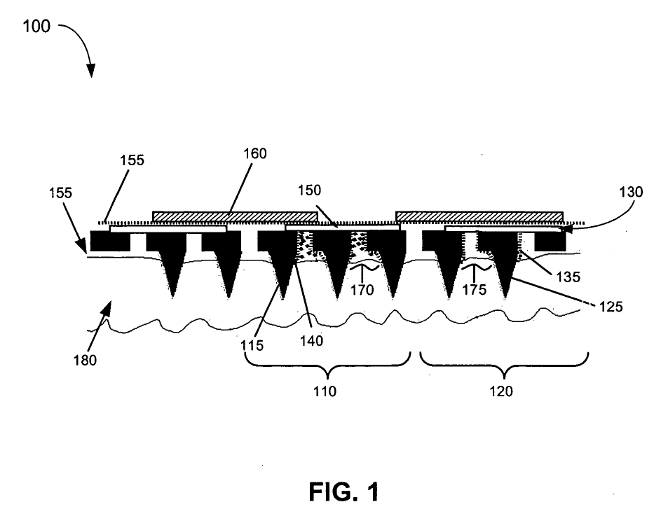

FIG. 1 shows a side view of biosensor array device 100 for the screening of a substance in a mammalian body, in accordance with an embodiment of the present invention. In one example, biosensor array 100 includes a reference region 120 and at least one sensor region 110. Reference region 120 includes at least one microneedle 125 and at least one reference microchannel electrode 130. The area between two adjacent reference microneedles 125 in reference region 120 defines the reference microchannels 175. Sensor region 110 includes at least one sensor microneedle 115 and at least one sensor microchannel electrode 150. The area between two adjacent sensor microneedles 115 in sensor region 110 defines the sensor microchannels 170.

Reference microneedle 125 has surface 135 that is free of ligands, and therefore cannot bind substance whether it is present or not in the mammal's body. Sensor microneedle 115 has surface 140 has been modified with a specific assay ligand, that lies within sensor microchannels 170, that will specifically bind to a given substance. Alternatively, in an electrode that is populated with a plurality of different ligand channels, the unbound channels could serve as reference against the active complexed ligand—substance channel.

Microneedles 115 and 125 are attached directly or indirectly to electrodes 150 and 130, respectively, and to support member 155. According to one embodiment, microneedles microchannels 170 and 175 are arranged or clustered with respect to corresponding electrodes 150 and 130, respectively. For example, the pitch or separation distance between adjacent microneedles 115 is less than the pitch or separation distance between adjacent electrodes 150.

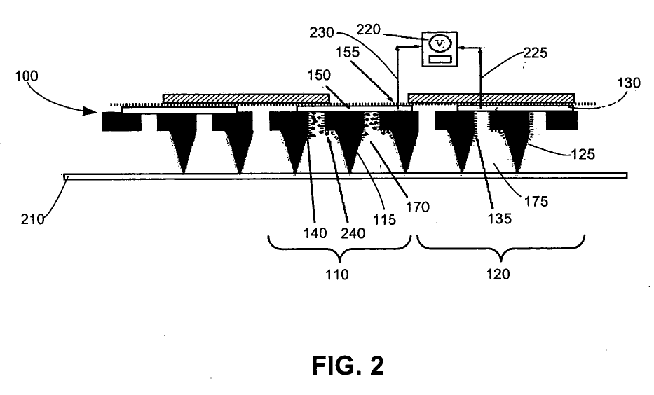

FIG. 2 shows an example implementation of the determination of the presence or absence of a substance in a mammalian body, in accordance with an embodiment of the present invention. FIG. 2 shows biosensor array 100, in contact with grounding electrode 210. Microchannels 170, which have substance bound to ligand 240 on surface 140, will include a higher resistance to electrical current flow compared to microchannels without the bound substance, i.e. 175. Since each electrode, e.g. 130 and 150, uses electromotive force to drive a specific current through respective microchannels, 175 and 170, electrodes attached to the top of the microneedles (e.g. 130 and 150) will exhibit differential voltages. Each differential voltage correlates with the presence or absence of specific substances that are the target of the ligand in that particular microchannel. Such measurements can be further refined by use of frequency domain dc or ac fluctuations to measure capacitive and dielectric components of the alterations created within the complexed ligand-molecule microchannel. Nevertheless, a device having this configuration will yield an immediate reading as to the presence or absence of a specific substance in the serum/bloodstream of the mammal subject.

In one embodiment, measuring device 220 is a voltmeter to measure the voltage difference between electrodes 150 and 130. If the ligand bonds with the particular substance to be sensed, the resistance against the current flow through the ligand-filled microchannel will increase; this increase will concurrently increase the voltage of electrode 150 with respect to ground. Biosensor array 100 is in contact with a grounding electrode 210. Voltmeter 220 is connected to electrode 150 via wire 230; voltmeter 220 is connected to electrode 130 via wire 225. Therefore, the voltage between electrode 150 and grounding electrode 210 and between electrode 130 and grounding electrode 210 can be measured by the voltmeter. If the measuring device reads a voltage difference, beyond a reasonable amount of error and beyond that resistance caused by the ligand itself, then it is apparent that the resistance of the ligand was increased and the substance to be detected is present; if no substance was present, then the two measured microchannels should have similar properties, and no voltage difference, and thus no additional resistance in the ligand, should have been detected.

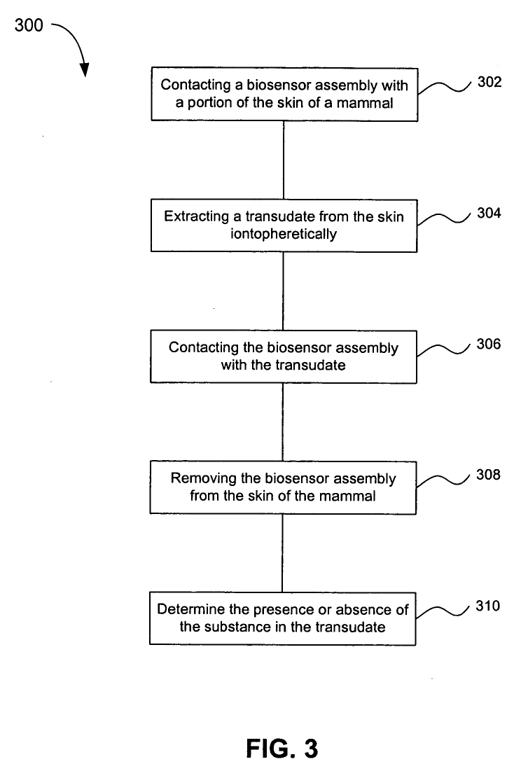

In another embodiment, the present invention relates to a method of screening a mammal for the presence or absence of a substance. FIG. 3 shows flowchart 300, which illustrates the steps involved in this method. Flowchart 300 begins with step 302, in which biosensor array 100 is put into contact with a portion of the skin of a mammal. In one example, microneedles are disposed in the stratum comeum of the subject mammal such that the microchannels of the biosensor array are disposed near the outer stratum comeum surface to facilitate contact with extracted transudate and limit the pain felt by the subject. Flowchart 300 continues with step 304, where a transudate is extracted from the skin of the subject iontophoretically. Step 306 follows step 304, in which the extracted transudate is contacted with the biosensor array. In one example, a current is applied to the electrodes of the biosensor array to drive the transudate across the stratum comeum and into contact with the biosensor array. The extracted transudate enters the microchannels and interacts with the surfaces of the microchannels. If a given substance is present in the transudate, and a microchannel surface has been modified with ligand that will bind the substance, the substance will bind to the ligand and remain bound to the surface of the microchannel. Flowchart 300 continues with step 308, in which the biosensor array is removed from the skin of the subject mammal. Finally, in step 310, the presence or absence of the substance is detected in the extracted transudate.

In one example, to detect the presence or absence of the substance, the biosensor array in contacted with a grounding electrode. In one example, the microneedles are contacted directly with the surface of the grounding electrode. A measuring device is coupled to the biosensor array. The microchannels that have substance bound to ligand deposited on the microchannel surface offer increased resistance to electrical flow as compared to microchannels without the bound substance. Since each electrode uses electromotive force to drive a specific current through such channels the electrodes will exhibit differential voltages. In one example, this differential voltage is measured by the device and correlates with presence or absence of specific substance that is the target of ligands in that particular microchannel. In another example, such measurements can be further refined by use of frequency domain dc or ac fluctuations to measure capacitive and dielectric components of the alterations created within the complexed ligand-molecule microchannel. In yet another example, localized capacitive loading can be used to determine the presence or absence of the substance. In another example, the presence or absence of the substance(s) can be measured by resonant frequency as modulated by the dielectric properties of the microchannels.

Substances that can be detected by the devices and methods of the present invention is any substance that can be extracted in the mammal transudate. For example, illicit drugs such as cocaine, heroin, cannabinoids, lysergic acid diethylamide, buprenorphine, methadone, barbiturates, benzodiazepines and other pyschoactive drugs can be detected. Also, the presence of certain pharmaceuticals can also be detected, including, but not limited to anabolic androgenic steroids and beta-2-agonists, toxic anticancer drugs, or other drugs.

The biosensor array of the present invention allows for greatly enhanced transudation of serum through the skin of the mammal subject. The transudate is expected to include peptide and protein components. The extraction is rapid and painless. In one example, the entire method can be completed in under two minutes. The use of the piezoelectric elements to generate ultrasound during the iontophoretic extraction is expected to greatly increase the effectiveness of the transudate extractions and help with the reproducibility and reliability of the reading by facilitating the motion of non complexed molecules that may nevertheless interfere with regional iontophoretic flow. In the extraction step the ultrasound may help with more uniform dispersion of the acquired transudate.

While not wishing to be bound by any one particular theory of operation, it is believed that catatonic forces are applied by the use of the ultrasound that greatly increases the effectiveness of the extraction.

In another embodiment of the present invention, multiple sensor regions are applied in an array of microneedles in a given biosensor so several substances can be the screened simultaneously.

### CONCLUSION

Exemplary embodiments of the present invention have been presented. The invention is not limited to these examples. These examples are presented herein for purposes of illustration, and not limitation. Alternatives (including equivalents, extensions, variations, deviations, etc., of those described herein) will be apparent to persons skilled in the relevant art(s) based on the teachings contained herein. Such alternatives fall within the scope and spirit of the invention. Thus, the breadth and scope of the present invention should be defined only in accordance with the following claims and their equivalents.

What is claimed is:

1. A screening device that screens for the presence or absence of one or more substances in a mammalian body, comprising:
a plurality of electrodes; and
a biosensor assembly having a reference region and at least one sensor region, wherein the reference region includes at least one microneedle and a microchannel, and each sensor region includes at least one microneedle and microchannel, and is sensitive to a respective substance,
wherein each sensor microchannel in a respective sensor region includes a specific assay ligand that may bond with the particular substance to be sensed.

2. The screening device of claim 6, further comprising:
a measurement device coupled to the plurality of electrodes to detect differences in electrical characteristics between the at least one microchannel at the reference region and the at least one microchannel at the sensor region indicative of the presence of respective one or more substances being screened.

3. A method of screening a mammal for the presence or absence of a substance, comprising:
contacting a biosensor assembly with a portion of the skin of a mammal;
extracting a transudate from the skin iontophoretically;
contacting the biosensor assembly with the transudate;
removing the biosensor assembly from the skin of the mammal; and
determining the presence or absence of the substance in the transudate;
wherein the biosensor assembly comprises a reference region and at least one sensor region, wherein the reference region includes at least one microneedle and a microchannel, and each sensor region includes at least one microneedle and microchannel, and is sensitive to the substance; and
wherein each sensor microchannel in a respective sensor region includes a specific assay ligand that may bond with the substance to be sensed.

4. The method of claim 3, further comprising applying ultrasound while performing said extracting step.

5. The method of claim 3, further comprising applying ultrasound while performing said determining step.

6. The device of claim 2, wherein the differences in electrical characteristics are voltage differences.

7. The method of claim 3, wherein the determining step comprises localized capacitive loading.

8. The method of claim 3, wherein the determining step comprises measuring resonant frequency as modulated by the dielectric properties of the microchannels.
